SEQUENCE LISTING (8 sequences)

SEQ ID NO: 1 - DNA, 1497 bp, Influenza A virus
atggcgtcccaaggcaccaaacggtcttatgaacagatggaaactgatggggatcgccagaatgcaactgagattagggcatccgtcgggaagatgattgatggaattgggcgattctacatccaaatgtgcactgaacttaaactcagtgattatgaagggcggttgatccagaacagcttgacaatagagaaaatggtgctctctgcttttgatgagagaaggaatagatatctggaagaacaccccagcgcggggaaagatcctaagaaaactggagggcccatatacaagagagtagatggaagatggatgagggaactcgtcctttatgacaaagaagaaataaggcgaatctggcgacaagccaacaatggtgaggatgcgacagctggtctaactcacatgatgatctggcattccaatttgaatgatacaacataccagaggacaagagctcttgttcgcaccggaatggatcccagaatgtgctctctgatgcagggctcgactctccctagaaggtctggagctgcaggtgctgcagtcaaaggaatcgggacaatggtgatggagctgatcagaatggtcaaacgggggatcaacgatcgaaatttctggagaggtgagaatgggcggaaaacaaggagtgcttatgagagaatgtgcaacattcttaaaggaaaatttcaaacagctgcacaaagagcaatggtggatcaagtgagagaaagtcggaacccaggaaatgctgagatcgaagatctcatatttttggcaagatctgcattaatattgagagggtcagttgctcacaaatcttgcctacctgcctgtgtgtatggacctgcagtatccagtgggtacgacttcgaaaaagagggatattccttggtgggaatagaccctttcaaactacttcaaaatagccaagtatacagcctaatcagaccgaacgagaatccagcacacaagagtcagctggtatggatggcatgccattctgctgcatttgaagatttaagattgttaagcttcatcagagggaccaaagtatctccgcgggggaaactttcaactagaggagtacaaattgcttcaaatgagaacatggataatatgggatcaagtactcttgaactgagaagcgggtactgggccataaggaccaggagtggaggaaacactaatcaacagagggcctccgcaggccaaatcagtgtgcaacctacgttttctgtacaaagaaacctcccatttgaaaagtcaaccgtcatggcagcattcactggaaatacggagggaagaacctcagacatgagggcagaaatcataagaatgatggaaggtgcaaaaccagaagaagtgtctttccgtgggcggggagttttcgagctctcagacgagaaggcaacgaacccgatcgtgccctcttttgacatgagtaatgaaggatcttatttcttcggagacaatgcagaagagtacgacaattaa

SEQ ID NO: 2 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 3 - protein, 502 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMDDMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFDDLARSALILRGSVAHKSCLPACVYGPAVASGYDFEREGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 4 - protein, 502 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMEDDLVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSQVYSLIRPNENPAHKSQLVDDWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 5 - DNA, 2420 bp, Influenza A virus
ggatccaggccctgccaggaaaaatataagggccctgcgtgagaacagagggggtcatccactgcatgagagtggggatgtcacagagtccagcccaccctcctggtagcactgagaagccagggctgtgcttgcggtctgcaccctgagggcccgtggattcctcttcctggagctccaggaaccaggcagtgaggccttggtctgagacagtatcctcaggtcacagagcagaggatgcacagggtgtgccagcagtgaatgtttgccctgaatgcacaccaagggccccacctgccacaggacacataggactccacagagtctggcctcacctccctactgtcagtcctgtagaatcgacctctgctggccggctgtaccctgagtaccctctcacttcctccttcaggttttcaggggacaggccaacccagaggacaggattccctggaggccacagaggagcaccaaggagaagatctgtaagtaggcctttgttagagtctccaaggttcagttctcagctgaggcctctcacacactccctctctccccaggcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttcactggctcttttcttctccatgcactgagcatctgctttttggaaggccctgggttagtagtggagatgctaaggtaagccagactcatacccacccatagggtcgtagagtctaggagctgcagtcacgtaatcgaggtggcaagatgtcctctaaagatgtagggaaaagtgagagaggggtgagggtgtggggctccgggtgagagtggtggagtgtcaatgccctgagctggggcattttgggctttgggaaactgcagttccttctgggggagctgattgtaatgatcttgggtggatcc

SEQ ID NO: 6 - protein, 309 aa, Influenza A virus
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 7 - protein, 311 aa, Influenza A virus
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLDDFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 8 - protein, 311 aa, Influenza A virus
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLDDIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

(19) United States
(12) Patent Application Publication
(10) Pub. No.: US 2004/0180058 A1
(43) Pub. Date: Sep. 16, 2004

(54) Vaccine compositions and methods

(76) Inventors: Alexander M. Shneider, Stoughton, MA (US); Michael Sherman, Newton, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/741,466

(22) Filed: Dec. 19, 2003

Related U.S. Application Data
(60) Provisional application No. 60/435,500, filed on Dec. 20, 2002.

(51) Int. Cl. A61K 39/00; A61K 39/38

(52) U.S. Cl. 424/184.1

(57) ABSTRACT

Methods of enhancing antigenic presentation or increasing immunogenicity of a polypeptide by modifying the three dimensional structure of a polypeptide.

### RELATED APPLICATIONS

This application claims the benefit of priority under 35 U.S.C. 119(e) to copending U.S. Provisional Application No(s). 60/435,500, filed on Dec. 20, 2002 as Docket No. 25955-003 PRO; the entire contents of which is incorporated herein by reference.

### FIELD OF THE INVENTION

The invention relates to vaccine compositions, methods of producing vaccine compositions, and methods of using these vaccines in treating cancer; cell proliferative; bacterial; and/or viral diseases such as influenza.

### BACKGROUND OF THE INVENTION

A vaccine is one of the most efficacious, safe, nontoxic and economical weapons to prevent disease and to control the spread of disease. Conventional vaccines are a form of immunoprophylaxis given before disease occurrence to afford immunoprotection by generating a strong host immunological memory against a specific antigen. The primary aim of vaccination is to activate the adaptive specific immune response, primarily to generate B and T lymphocytes against specific antigen(s) associated with the disease or the disease agent.

Certain viral diseases can currently be controlled, but efficacious and long-term prevention has not yet been obtained. For example, influenza is a contagious disease that is caused by the influenza virus. It attacks the respiratory tract in humans (nose, throat, and lungs). Influenza usually comes on suddenly and includes symptoms of, e.g., fever, headache, and dry cough. Most people who get influenza will recover in one to two weeks, but some people will develop life-threatening complications (such as pneumonia) as a result of the flu. Millions of people in the United States—about 10% to 20% of U.S. residents—will get influenza each year. An average of about 36,000 people per year in the United States die from influenza, and 114,000 per year have to be admitted to the hospital as a result of influenza. Serious problems from influenza can happen at any age, but particularly in the elderly, e.g., 65 years and older, people with chronic medical conditions; and very young children are more likely to get complications, e.g., pneumonia, bronchitis, and sinus and ear infections from influenza.

People with asthma may also experience asthma attacks while they have the flu, and people with chronic congestive heart failure may have worsening of this condition that is triggered by the flu.

Currently the flu shot, made from inactivated viruses, is available and is in widespread use. A better approach, however, would be the development of a DNA or protein-based vaccine which would induce a permanent immune response (rather than having to administer it yearly, like the flu shot), and which does not rely on inactivated viruses and the possible side effects of the use thereof, e.g., apprehensions about using same in pregnant women. Furthermore, the current flu vaccines have a disadvantage in that they are narrowly focused on one specific viral strain. A better vaccine would have a wide range of anti-flu protection covering many, if not all, strains.

A major hindrance to the development of effective T-cell based immunotherapies is that antigen presentation on the surface of cells is often inadequate to elicit a sufficient primary T-cell response to the antigen. Nevertheless, the amount of antigen presentation on the cell surface is adequate to elicit a secondary response, if the primary immune response is previously elicited. Thus, a major aim of researchers in fields such as cancer biology, virology and immunology is to develop treatment methods that enhance antigen presentation, which would allow for the formation of a primary immune response.

### SUMMARY OF THE INVENTION

The present invention is directed to new vaccine compositions, methods of producing them, and methods of using these vaccines in preventing and treating diseases, e.g., cancer; cell proliferative; bacterial; and/or viral, such as influenza. The invention features the novel concept of a viral DNA molecule coding for a disease-associated, e.g., viral, protein, or the viral protein itself, which contains a disruptive element in one or more regions internal to the protein in question. The “normal” viral protein in, or produced by, the cell is typically poorly presented due to the inability of the cell to sufficiently degrade the protein via the ubiquitin-proteasome degradation system into peptides which can bind to MHC-I and thus be presented on the cell surface for binding by a T cell, e.g., cytotoxic lymphocytes, with concomitant destruction of the infected cell(s). Presentation of similar peptides on MHC-I in specialized antigen presenting cells (e.g., dendritic cells) leads to development of a permanent immune response via activation of proliferation of the proper T-cell clones. By the introduction of the disruptive element, e.g., a deletion, substitution or insertion in the internal, e.g., hydrophobic, portions of the protein (or in the coding sequence for that protein), the conformation of that protein in the cell is changed so that the ubiquitin-proteasome system degrades the protein much more efficiently, resulting in more peptides that are generated and that bind more frequently to MHC-I, and therefore induce a more effective and long-term T cell response.

One aspect of the invention relates to methods of enhancing protein degradation, antigenic presentation or increasing the immunogenicity of a polypeptide by modifying the three dimensional structure of a polypeptide. The modification is a disruptive element in one or more inner (e.g., hydrophobic) domain regions of the polypeptide, which forces a conformational change in the protein structure, resulting in increased proteolytic degradation, e.g., in the proteasome. The disruptive element alters the tertiary structure of the modified viral protein as compared to unmodified viral protein, allowing for the increased degradation.

The disruptive element may be an insertion or deletion of one or more amino acids, or a substitution of one or more amino acids (e.g., a charged, or hydrophilic, amino acid for an uncharged or hydrophobic amino acid.) Advantageously, the disruptive element is an exogenous amino acid sequence containing two or more amino acids, e.g., two negatively charged amino acids such as aspartate residues.

Another aspect of the invention provides a method of inducing an immune response in a subject against a disease-associated, e.g., viral, protein by introducing a modified viral protein containing a disruptive element into the subject such that the immune response is induced. The disruptive element includes the insertion, deletion, and/or substitution of one or more amino acids of the protein. By way of non-limiting examples, the disruptive element includes one or more (e.g., 1, 2, 3, 4, 5, 7, 10 or more) hydrophilic amino acids (e.g., aspartate, asparagine, glutamate, histidine, lysine, or arginine) substituted for one or more hydrophobic amino acids (e.g., phenylalanine, cysteine, isoleucine, leucine, valine or tryptophan.) The hydrophilic amino acids may be contiguous, but alternatively, the hydrophilic amino acids may be discontiguous.

The disruptive element is located in an internal region of the amino acid sequence. The internal region of said amino acid sequence may be and is typically hydrophobic, but alternatively, the disruptive element may be located in an amphiphilic α-helical region.

In embodiments of the invention, the disruptive element is located at or near a terminus of the polypeptide sequence, e.g., the N-terminus or the C-terminus of the polypeptide sequence. “Near a terminus” includes amino acid positions within 1, 5, 10, 20, 50, 75 or 100 amino acids of the terminus. In preferred embodiments of the invention, the disruptive element is located in a domain structure of the viral protein. A domain structure of a (viral) protein includes any polypeptide that is at least one amino acid shorter in length than the protein. Domain structures are structures that affect the secondary structure of the polypeptide (e.g., alpha helical regions, beta pleated sheet regions, or coils.) Alternatively, domain structures are amino acid sequences that affect the protein, e.g., binding to a ligand, recognition by an antibody, catalytic activity, or binding with other molecules. Domain structures include, but are not limited to, PDZ, pleckstrin homology (PH), tec homology (TH), a proline-rich region, Src Homology 3 (SH3), and Src Homology 2 (SH2). One of ordinary skill in the art can identify suitable protein domains in a polypeptide of interest using domain databases such as Pfam (Pfam is a large collection of multiple sequence alignments and hidden Markov models covering many common protein domains and families. Pfam is accessible online from the Sanger Institute, UK, and other locations.) In an embodiment of the invention, a disruptive element includes two aspartate residues in close proximity to one another (e.g., within 1, 2, 3, 5, 10, 15 or more amino acid resides of one another.) The disruptive element may be inserted or be present in an extended alpha helical domain internal to the three dimensional structure of the protein. Alternatively, the two aspartate residues are in proximity to each other in the tertiary structure of the viral protein (e.g., the two aspartate residues are separated by less than about 1 to about 100 angstroms.)

In an especially advantageous embodiment, the invention relates to influenza vaccines, and the uses thereof, which are improved over those currently available. A DNA molecule (typically contained in a suitable vector) encoding a modified influenza NP protein (i.e., containing the disruptive element(s) as described herein), is delivered to a patient, which results in an enhanced, stable and wide ranging immune response. Influenza NP protein is highly conserved, so as such, an influenza vaccine of the invention will be effective on a wide range of (if not all) specific viral strains, an important benefit. In embodiments of the invention, the described vaccines, having a modified NP nucleic acid or a modified NP polypeptide, are administered in combination with one or more additional vaccines, e.g., vaccines that do not contain a modified NP nucleic acid or a modified NP polypeptide.

The modified protein (or nucleic acid encoding the modified protein) of the invention is associated with a disease or disorder. The modified protein is, e.g., a tumor-associated polypeptide, a cell proliferative disorder-associated polypeptide, or a disease-associated viral polypeptide. The viral polypeptide may be a core protein, such as the NP protein (i.e., a viral nuclear protein or nucleoprotein.)

The present invention provides modified disease-associated, e.g., viral, polypeptides capable of undergoing efficient proteolytic cleavage, including polypeptides that are degraded to one or more peptides of less than about 50, about 25, about 15, about 10 or about 5 amino acids in length. The modified viral polypeptide has altered susceptibility to proteolysis (e.g., proteasome-dependent or proteasome-independent proteolysis) as compared to an unmodified viral protein. The modified proteins of the invention include polypeptides that, when proteolytically processed, e.g., in the proteasome, generate one or more peptides that bind to a MHC class I molecule.

In another aspect, the present invention provides a vaccine that includes a nucleic acid molecule that encodes and is capable of expressing a modified viral protein that contains a disruptive element, in an amount effective to elicit an immune response. The nucleic acid encodes a modified viral protein that has altered susceptibility to proteolysis as compared to an unmodified viral protein. The nucleic acid molecule may be operably linked to a promoter. Further, the nucleic acid molecule may be in a vector, such as a vector capable of directing expression of a nucleic acid encoding a modified viral protein. In embodiments of the invention, the vectors may be a virally derived vector, such as a vaccinia virus vector, an RNA vector such as a retroviral vector, or a lentiviral vector. The invention also provides a method of immunization, that includes administering to a subject this vaccine. The subject may be a mammal (e.g., a human or non-human primate, dog, cat, pig, sheep, cow, horse, goat or rodent), suffering from or at risk of cancer, a viral infection or a disorder associated with improper gene expression. Alternatively, the invention provides a method of immunization, including the steps of providing a subject cell, contacting this cell with the vaccine, and administering this cell to a subject, such that the subject is immunized.

Administration may be by intraperitoneal, subcutaneous, nasal, intravenous, oral, topical or transdermal delivery. In embodiments of the invention the vaccine is administered in a vector (e.g., a DNA vector or RNA vector) or a liposome. In other embodiments, the vaccine is administered with one or more compounds, including compounds that increase antigen presentation, adjuvants, and cytokines, such as interferon-γ.

In a further aspect, the present invention relates to a method of inducing an immune response in a subject against a viral protein, which includes the steps of introducing into a subject a nucleic acid molecule encoding a modified viral protein that contains a disruptive element, where the nucleic acid molecule is capable of being expressed in a cell of the subject such that the immune response is induced.

The present invention also provides a vaccine that includes a vector containing a promoter operably linked to a nucleic acid molecule encoding a modified NP polypeptide that includes a disruptive element, in an amount effective to elicit an immune response. The promoter may be a cytomegalovirus (CMV) promoter or a vaccinia virus (VV)-P65 promoter, or other promoters known to those skilled in the art. In certain embodiments, the vector is a vaccinia virus vector.

In another aspect, the invention provides a method of forming a vaccine capable of stimulating the immune mechanism of a mammal, including the steps of introducing a disruptive element into a nucleic acid encoding a viral polypeptide to form a modified viral polypeptide, where this modified viral polypeptide has altered susceptibility to proteolysis as compared to an unmodified viral protein, and combining the modified viral polypeptide with a vaccine carrier, such that a vaccine is formed.

In another aspect, the invention provides a method of forming a vaccine capable of stimulating the immune mechanism of a mammal, comprising introducing a disruptive element into a viral polypeptide to form a modified viral polypeptide, wherein the modified viral polypeptide has altered susceptibility to proteolysis as compared to an unmodified viral protein, and combining the modified viral polypeptide with a vaccine carrier, such that a vaccine is formed.

The invention further provides a method of immunization in a subject, including the steps of providing a subject cell, contacting the cell with a vaccine containing a nucleic acid encoding a modified viral protein, and administering the cell to the subject, such that the subject is immunized thereby. In embodiments of the invention, the subject cell is isolated from the subject.

In another aspect, the present invention provides a method of generating a substantially pure population of educated, antigen-specific immune effector cells, including the steps of contacting immune effector cells with an antigen presenting cell, wherein the antigen presenting cell contains a nucleic acid molecule encoding a modified viral protein containing a disruptive element, when the modified viral protein is capable of being expressed in the antigen presenting cell. Alternatively, the invention provides a substantially pure population of educated, antigen-specific immune effector cells produced by culturing immune effector cells with an antigen presenting cell containing a nucleic acid molecule encoding a modified viral protein that includes a disruptive element, when the modified viral protein is capable of being expressed in the antigen presenting cell. The antigen-specific immune effector cells may be T lymphocytes.

The present invention also provides a method of inducing an immune response in a subject against a protein, including the steps of introducing a modified protein that contains a disruptive element and a modification site into the subject, such that the immune response is induced. The modification site is a site for a biological process. A biological process includes phosphorylation, dephosphorylation, glycosylation, acetylation, methylation, ubiquitination, sulfation, proteolysis, prenylation, and selenium incorporation, transglutamination, methylation, acetylation, SUMOylation. The biological process causes an alteration in the tertiary structure of said protein.

The invention relates to polypeptides that are improperly expressed in mammalian cells. For example, tumor cells produce tumor-specific antigens (TSAs) as well as tumor-associated antigens (TAAs, antigens that are associated with the onset and/or progression of cancer, which are expressed on tumor cells and non-tumor cells). Examples of tumor antigens include MAGE-1, MAGE-3, MART-1, gp100, tyrosinase, tyrosinase-related protein-1, BAGE, GAGE-1, GAGE-3, gp75, oncofetal antigen, mutant p53, mutant ras and telomerase. Further, improper protein folding is a critical factor in the development of various human diseases such as Alzheimer's Disease and cancer. (See Tjernberg et al., 1999. JBC 274:12619; Lim et al., 2001. J. Clin. Path. 54:642). Deregulation of expression and folding of the cellular prion protein (PrP_{c}) and its conversion into its pathological isoform (PrP_{Sc}) is associated with human and veterinary diseases.

The invention relates, in part, to modified viral polypeptides. Non-limiting examples of modified viral polypeptides are the modified influenza NP polypeptides provided in Table 1.

The invention also relates to modified nucleic acid molecules. A non-limiting example is a nucleic acid molecule that includes a nucleic acid sequence encoding the amino acid sequence of a modified influenza NP polypeptide.

In another aspect, the invention provides a method for presentation of antigens, including the steps of contacting an antigen presenting cell with a nucleic acid molecule that encodes a viral polypeptide having a disruptive element in an internal region of the peptide, and causing the nucleic acid molecule to be expressed in the antigen presenting cell, such that one or more peptides derived from the viral polypeptide are presented as antigens by the antigen presenting cell. These one or more derived peptides are associated with MHC class I molecules.

The invention further provides a method for formulation of a vaccine, including the steps of providing an amino acid sequence encoding a viral protein, identifying one or more amino acids of the viral polypeptide suitable for deletion or replacement, or as an insertion point for introduction of a disruptive element, such that the disruption alters the tertiary structure of the viral polypeptide, and introducing the disruptive element into a nucleic acid sequence encoding the viral protein, wherein the nucleic acid is capable of being expressed, whereby a vaccine is formulated.

In its various aspects the invention also provides a vaccine in an amount effective to elicit an immune response, e.g., T-cell or B-cell. The vaccine contains a nucleic acid molecule encoding a modified polypeptide. The modified polypeptide has an altered three-dimensional structure, increased antigen presentation and/or increased proteolytic degradation compared to a corresponding unmodified polypeptide when the nucleic acid molecule is expressed in a cell. The modification is, for example, an insertion or deletion of one or more amino acids and/or amino acid sequences. Preferably, the modified polypeptide has increased degradation to a peptide. No particular length is implied by the term peptide. The peptide can bind MHC class I molecules.

The nucleic acid is DNA or RNA. The nucleic acid contains the coding region of the polypeptide. The nucleic acid is operably linked to a promoter, e.g., CMV, RSV, EF-1a, or SV40 promoter. In some aspects the nucleic acid is in a vector, such a vaccine virus vector. Alternatively, the nucleic acid is in a plasmid, or delivered by itself or in a liposome. The polypeptide is a viral polypeptide, such a core protein. The core protein is a NP protein of influenza virus. Alternatively, the polypeptide is a tumor-associated polypeptide, a cell proliferative disorder associated polypeptide or a bacterial polypeptide.

Also provided by the invention is a method of immunization by administering to a subject, e.g., human the vaccine according to the invention. Immunization is in vivo or alternatively, ex vivo. The subject is further administered a compound that increases antigen presentation such as gamma interferon or a cytokine. Administration is prophylactic or alternatively therapeutic. In some aspects the subject is suffering from or at risk of cancer, a viral infection or a disorder associated with improper gene expression, e.g., a cell proliferative disorder. Administration may be intraperitoneal, subcutaneous, nasal, intravenous, oral, topical or transdermal in a vector, e.g. viral vector, DNA vector, or an RNA vector or a liposome.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a set of photographic images of NP proteins having FLAG tags resolved by polyacrylamide gel electrophoresis (PAGE) and blotted with an anti-FLAG antibody. FIG. 1a demonstrates the reduced levels of full-length modified NP protein following treatment with cycloheximide (CDI). FIG. 1b shows that proteolysis of unmodified NP is blocked upon inhibition of proteasome by MG132 treatment.

### DETAILED DESCRIPTION OF THE INVENTION

Classic vaccination is aimed toward developing a B-cell immune response to a pathogen, e.g., virus, bacteria or tumor associated antigen. Vaccines are administered as a preventative measure to an organism to elicit neutralizing antibodies to the pathogen. When a pathogen infects the organism later on, the antibodies bind the pathogen and eliminate it from the organism. This approach underlies every successful vaccine developed, e.g., smallpox. However, there are viral pathogens such as influenza, which are resistant to B-cell based vaccination. Their surface proteins mutate rapidly, thus escaping the antibody response, as the antibodies can no longer recognize the mutated virus with altered surface proteins. A further limitation of classic vaccination is that it is preventative only and cannot be used therapeutically after the pathogen has infected an organism.

The invention is based in part on an alternative to classic vaccination, by activating the T-cell branch of immune system to target an infected cell rather than the pathogen, e.g., viral particles in the serum. Infected cells present peptides derived from pathogen proteins on their surface in complex with MHC-I proteins. If the number of pathogen-derived peptides presented on the cell surface exceeds a threshold, propagation of a specialized clone of T-cells that specifically recognizes the infected cells is induced, and eliminates infected cells. Multiple mechanisms have evolved in viruses that prevent or reduce T-cell immune response. One critical and ubiquitous mechanism is the acquisition by viral proteins of a structure that prevents their degradation by proteasomes and thus reduces their processing and generation of peptides to be presented on MHC-1. For example, NP-protein (nuclear protein or nucleoprotein) of influenza virus is poorly processed by the cellular proteolytic machinery, leading to its poor presentation on MHC-1 and poor activation of T-cell immune response. Influenza NP has a lower rate of mutation as compared to influenza surface proteins (see, e.g., Lee et al., 2001. Arch. Virol. 146:369-77). Influenza nucleoprotein (Influenza A/Puerto Rico/8/34 strain) contains an H-2 Kd-restricted CD8+ T cell (T CD8+) epitope spanning amino acid residues 147-155. It has been demonstrated that expression of NP147-155 and NP147-158 in isolation via “minigene”/recombinant vaccinia virus (vac) technology leads to sensitization of target cells for NP-specific killing while expression of 147-158 lacking the arginine at position 156 (termed here as 147-155TG) does not, and that addition of a single amino acid, Met159, to the C terminus of the blocked peptide (creating 147-155TGM) restores presentation. (See, Yellen-Shaw, et. al., 1997 J Immunol. 158(4):1727-33).

In its various aspects the invention provides a method of modifying, e.g., increasing, enhancing, or reducing antigen presentation or immunogenicity of a polypeptide by modifying the three-dimensional structure or proteolytic degradation of the polypeptide as compared to a corresponding non-modified (i.e., control) polypeptide.

The invention also provides a vaccine having in an amount effective to elicit an immune response a nucleic acid encoding a modified protein or polypeptide, e.g., a tumor-associated polypeptide, a cell proliferative disorder-associated polypeptide, or a disease-associated viral polypeptide. The modified polypeptide has an altered three-dimensional structure, increased proteolytic degradation or increased antigen presentation compared to an unmodified polypeptide, when expressed in a cell.

Definitions

A “viral protein” includes any polypeptide encoded by a viral gene. As used herein, “polypeptide” and “protein” are synonymous.

A “disease-associated protein” includes a polypeptide whose expression, cell or tissue localization, or folding is associated with one or more diseases and also includes viral conditions like influenza. Tumor specific antigens (TSAs) and tumor-associated antigens (TAAs) are exemplary disease-associated proteins.

A “modified viral protein” includes a viral protein that has a different primary, secondary or tertiary amino acid sequence as compared to a unmodified viral protein (i.e., a wild-type viral protein.)

A “modified nucleic acid” or “modified viral nucleic acid” includes a nucleic acid that encodes for a modified (viral) protein.

A “disruptive element” includes any modification to a viral protein or to a nucleic acid encoding a modified viral protein that disrupts the three dimensional structure of the protein, such that the proteolytic degradation of the modified viral protein is altered (e.g., increased or decreased.) Such modification includes an insertion, substitution or deletion of one or more amino acids, or an insertion, substitution or deletion of one or more nucleic acids in a nucleic acid sequence that encodes a viral protein, preferably in an internal, e.g., hydrophobic region of the protein.

The “tertiary structure” of a polypeptide represents the three-dimensional structure of a polypeptide.

The “secondary structure” of a polypeptide represents the folding of the peptide chain into an alpha helix, beta pleated sheet, or random coil. The secondary structure of a polypeptide can be determined by applying one or more algorithms to the primary amino acid sequence of the polypeptides. These algorithms include the DPM method, the Homolog method, and the Predator method.

A “domain structure” of a viral protein includes any polypeptide derived from the viral protein that is at least one amino acid shorter in length than the viral protein. Generally, domain structures are structures that define the secondary structure of the polypeptide or affect the activity of the polypeptide binding to a ligand, recognition by an antibody, catalytic activity, or binding with other molecules.

An “internal region” of a polypeptide includes any amino acid of the polypeptide other than the N-terminal or C-terminal amino acid. An internal region of a polypeptide also includes one or more amino acids present in a hydrophobic domain of a polypeptide.

A “hydrophobic domain” of a polypeptide includes regions of the polypeptide that are inaccessible to solvent under physiological (e.g., non-denaturing) conditions.

A “tumor-associated polypeptide” includes polypeptides that are associated with the onset and/or progression of tumor growth or cancer cell proliferation.

A “cell proliferative disorder” includes cancer, restenosis, retinopathy and other vasoproliferative diseases.

“Antigen presentation” includes the expression of antigen on the surface of a cell in association with major histocompatability complex class I or class II molecules (MHC-I or MHC-IL) Antigen presentation is measured by methods known in the art. For example, antigen presentation is measure using an in vitro cellular assay as described in Gillis, et al., J. Immunol. 120: 2027 (1978).

“Immunogenicity” includes the ability of a substance to stimulate an immune response. Immunogenicity is measured, for example, by determining the presence of antibodies specific for the substance. The presence of antibodies is detected by methods known in the art, for example an ELISA assay.

“Proteolytic degradation” includes degradation of the polypeptide by hydrolysis of the peptide bonds. No particular length is implied by the term peptide. Proteolytic degradation is measured, for example, using electrophoresis (e.g., gel electrophoresis), NMR analysis or mass spectral analysis. As used herein, “cancer” includes any abnormal cell proliferation, including invasive and non-invasive tumors.

As used herein, a “virus” includes any infectious particle having a protein coat surrounding an RNA or DNA core of genetic material.

As used herein, “autoimmune disease” includes any disease or disorder characterized by or involving autoimmune antibodies or lymphocytes that attack molecules, cells, or tissues of the organism producing them, e.g., lupus, rheumatoid arthritis, multiple sclerosis, systemic sclerosis, diabetes mellitus, Rasmussen's encephalitis, Lambert Eaton Myasthenic Syndrome, myasthenia gravis, tropical spastic paraperesis/HTLV-1-associated myelopathy (TSP/HAM), autoimmune peripheral neuropathies, chronic inflammatory demyelinating polyneuropathy (CIDP), autoimmune cerebellar degeneration, opsoclonus/myoclonus (Anti-Ri), stiff person syndrome, and gait ataxia with late age onset polyneuropathy (GALOP).

By a “portion” of the polypeptide is meant two or more amino acids of the polypeptide, and include domains of the polypeptide (e.g., the intracellular, transmembrane or extracellular domains, signal peptides, and nuclear localization signals.) A portion includes any fragment of a polypeptide created by proteolytic cleavage.

The cell may be any cell capable of antigen presentation. Antigen presenting cells (APCs) capture and process antigens for presentation to T-lymphocytes, and produce signals required for the proliferation and differentiation of lymphocytes. APCs include somatic cells, B-cells, macrophages and dendritic cells (e.g., myeloid dendritic cells.)

Modified Viral Polypeptides

The present invention relates, in part, to modified viral polypeptides (and nucleic acids encoding them for expression in cells) that contain a disruptive element in the polypeptide sequence. The disruptive element results in a conformational change in the modified polypeptide structure, such that the proteolytic processing of the modified polypeptide is different from that of the unmodified polypeptide. Without wishing to be bound by theory, one mechanism of action for the difference in proteolytic processing is that the conformational change alters (e.g., increases or decreases) the accessibility of internal amino acids. Proteolytic processing occurs via the proteasome. Alternatively, proteolytic processing occurs via non-proteasomal pathways.

Preferred modified viral polypeptides include modified influenza NP polypeptides, non-limiting examples of which are provided in Table 1.

TABLE 1

Modified NP polypeptides

| | Corresponding | | |
|---|---|---|---|
| | amino acids of | | |
| Target NP peptide | SEQ ID NO: 2 | Amino acid substitutions¹ | Amino acid Insertions² |

| | | | |
|---|---|---|---|
| FYIQMCT | 39-45 | ³⁹FYDQMCT⁴⁵ | ³⁹FDDYIQMCT⁴⁵ |
| | | ³⁹FYIQDDT⁴⁵ | |
| SLTI | 60-63 | ⁶⁰SUTI⁶³ | ⁶⁰SDDLTI⁶³ |
| RRIWR | 117-121 | ¹¹⁷RRDDR¹²¹ | ¹¹⁷RDDRIWR¹²¹ |
| TMVMELVRMIKR | 188-199 | ¹⁸⁸TMVMEDDRMIKR¹⁹⁹ | ¹⁸⁸TMVMEDDLVRMIKR¹⁹⁹ |
| | | ¹⁸⁸TMVMELVRDDKR¹⁹⁹ | |
| NAEFEDLTFLARSALIL | 250-270 | ²⁵⁰NAEFEDLTDDARSALI | ²⁵⁰NAEFEDLTFDDLARS |
| RGSV | | LRGSV²⁷⁰ | ALILRGSV²⁷⁰ |
| | | ²⁵⁰NAEFEDLTFLARSADD | |
| | | DRGSV²⁷⁰ | |
| QLVWMACHSAAFE | 327-339 | ³²⁷QLVDDACHSAAFE³³⁹ | ³²⁷QLVDDWMACHSAAFE³³⁹ |
| | | ³²⁷QLVWDDCHSAAFE³³⁹ | ³²⁷QLVWMACHSAADDFE³³⁹ |
| | | ³²⁷QLVWMDDHSAAFE³³⁹ | |
| | | ³²⁷QLVWMACHSADDE³³⁹ | |
| MRTEIIRMMES | 440-450 | ⁴⁴⁰MRTEDDRMMES⁴⁵⁰ | ⁴⁴⁰MRTEDDIIRMMES⁴⁵⁰ |
| | | ⁴⁴⁰MRTEIIRDDES⁴⁵⁰ | |

¹Substituted amino acids are in bold and underlined.
²Inserted amino acids are in bold and underlined.

The disruptive element may be an insertion or deletion of one or more amino acids, or a substitution of one or more amino acids (e.g., a charged, or hydrophilic, amino acid for an uncharged or hydrophobic amino acid.) Alternatively, the disruptive element is an exogenous amino acid sequence containing two or more amino acids that are capable of being acted upon by a protease, or a combination of two or more proteases. A non-limiting example is the insertion of the sequence DEVDG into a polypeptide (e.g., between two amino acids, neither of which are at the N- or C-terminus.) This sequence includes a cleavage site for the caspase-3 protease, where the protease cleaves the peptide between the C-terminal D and the G. Useful proteases or proteolytic enzymes include Arg-C proteinase, Asp-N endopeptidase, BNPS_Skatole, Caspase1, Caspase2, Caspase3, Caspase4, Caspase5, Caspase6, Caspase7, Caspase8, Caspase9, Caspase10, Chymotrypsin (e.g., high specificity (C-term to [FYW], not before P) or low specificity (C-term to [FYWML], not before P)), Clostripain, Enterokinase, GranzymeB, Factor Xa, Glutamyl endopeptidase, Pepsin, Proline-endopeptidase, Proteinase K, Staphylococcal peptidase I, Thermolysin, Thrombin and Trypsin. For example, pepsin preferentially cleaves at Phe, Tyr, Trp and Leu in position P1 or P1′ of the peptide. Protease cleavage sites are generally known in the art, using programs such as Peptide Cutter (available on the ExPASy (Expert Protein Analysis System) proteomics server of the Swiss Institute of Bioinformatics (SIB))

The disruptive elements described herein (such as one or more aspartate-aspartate (DD) dipeptides inserted into the polypeptide sequence of the NP polypeptide sequence) increase proteolytic degradation of the modified polypeptide, which increases antigen presentation by antigen-presenting cells (APCs) when the modified polypeptides are introduced into a mammalian subject, thereby increasing the immune response of the subject to the polypeptide.

A disruptive element can be introduced into a polypeptide to form a modified polypeptide by introducing the disruptive element directly into the polypeptide, or introducing the disruptive element into the nucleotide sequence encoding the polypeptide, whereby translation of the nucleic acid sequence results in a polypeptide containing the disruptive element.

Introduction of a Disruptive Element Into a Nucleic Acid Encoding a Modified Polypeptide.

A modified polypeptide may be generated by expressing a modified polypeptide encoded by a modified nucleic acid, or by directly modifying the polypeptide. Modifying the three-dimensional structure of a polypeptide is accomplished, for example, by modifying the amino acid sequence by inserting or deleting one or more amino acids in the polypeptide sequence such that the three-dimensional structure of the polypeptide is altered, i.e., including the disruptive element. The disruptive element is located in an internal region of the polypeptide, e.g., in a domain structure like an extended α-helical domain. For example, an amino acid sequence of 1, 3, 5, 10, 25, 50, 100 or more amino acids is inserted or deleted. Alternatively, one or more amino acids in the polypeptide sequence of the unmodified polypeptide are substituted by one or more different amino acids. Alternatively, modification of the three-dimensional structure is accomplished by inserting or deleting an amino acid sequence of 1, 3, 5, 10, 25, 50, 100 or more amino acids within a domain structure of the polypeptide. Modification is at the protein level. Alternatively, modification is at the DNA or RNA level, e.g., inserting or deleting one or more nucleic acids in an unmodified nucleotide sequence encoding the unmodified polypeptide, thus generating a modified nucleotide sequence encoding a modified polypeptide, or substituting one or more nucleic acids for one or more different nucleic acids.

The position wherein the disruptive element is introduced into the amino acid sequence impacts the effect of the disruptive element on proteolysis. Preferably, the disruptive element is introduced at one or more inner hydrophobic domain regions of the polypeptide.

The modification to the polypeptide results in a conformational change in the polypeptide such that the proteolytic degradation of the modified polypeptide is altered, i.e., increased, relative to the unmodified peptide, e.g., the modified polypeptide is more efficiently proteolytically processed, or the modified polypeptide is a substrate for one or more proteolytic enzymes that do not act upon the unmodified polypeptide.

The polypeptide is, for example, a viral peptide, such a viral core protein, e.g., the NP protein of influenza; a bacterial protein; a tumor-associated protein; or a polypeptide associated with aberrant gene expression. An influenza NP nucleic acid and polypeptide sequences are shown in Table 2. Influenza NP nucleic acids include, e.g., GenBank Accession Numbers AB126632, AF536708, AJ293924, and AF483604. Influenza NP amino acids include, e.g., GenBank Accession Numbers NP_{—}775533, CAA91084, and P31609. Non-limiting examples of modified NP nucleic acid and amino acid sequences are provided in Table 3 and in the Examples section.

TABLE 3
Influenza NP nucleic acid and polypeptide sequences

| | | | | | |
|---|---|---|---|---|---|
| atggcgtccc | aaggcaccaa | acggtcttat | gaacagatgg | aaactgatgg | ggatcgccag |
| aatgcaactg | agattagggc | atccgtcggg | aagatgattg | atggaattgg | gcgattctac |
| atccaaatgt | gcactgaact | taaactcagt | gattatgaag | ggcggttgat | ccagaacagc |
| ttgacaatag | agaaaatggt | gctctctgct | tttgatgaga | gaaggaatag | atatctggaa |
| gaacacccca | gcgcggggaa | agatcctaag | aaaactggag | ggcccatata | caagagagta |
| gatggaagat | ggatgaggga | actcgtcctt | tatgacaaag | aagaaataag | gcgaatctgg |
| cgacaagcca | acaatggtga | ggatgcgaca | gctggtctaa | ctcacatgat | gatctggcat |
| tccaatttga | atgatacaac | ataccagagg | acaagagctc | ttgttcgcac | cggaatggat |
| cccagaatgt | gctctctgat | gcagggctcg | actctcccta | gaaggtctgg | agctgcaggt |
| gctgcagtca | aaggaatcgg | gacaatggtg | atggagctga | tcagaatggt | caaacggggg |
| atcaacgatc | gaaatttctg | gagaggtgag | aatgggcgga | aaacaaggag | tgcttatgag |
| agaatgtgca | acattcttaa | aggaaaattt | caaacagctg | cacaaagagc | aatggtggat |
| caagtgagag | aaagtcggaa | cccaggaaat | gctgagatcg | aagatctcat | atttttggca |
| agatctgcat | taatattgag | agggtcagtt | gctcacaaat | cttgcctacc | tgcctgtgtg |
| tatggacctg | cagtatccag | tgggtacgac | ttcgaaaaag | agggatattc | cttggtggga |
| atagaccctt | tcaaactact | tcaaaatagc | caagtataca | gcctaatcag | accgaacgag |
| aatccagcac | acaagagtca | gctggtatgg | atggcatgcc | attctgctgc | atttgaagat |
| ttaagattgt | taagcttcat | cagagggacc | aaagtatctc | cgcgggggaa | actttcaact |
| agaggagtac | aaattgcttc | aaatgagaac | atggataata | tgggatcaag | tactcttgaa |
| ctgagaagcg | ggtactgggc | cataaggacc | aggagtggag | gaaacactaa | tcaacagagg |
| gcctccgcag | gccaaatcag | tgtgcaacct | acgttttctg | tacaaagaaa | cctcccattt |
| gaaaagtcaa | ccgtcatggc | agcattcact | ggaaatacgg | agggaagaac | ctcagacatg |
| agggcagaaa | tcataagaat | gatggaaggt | gcaaaaccag | aagaagtgtc | tttccgtggg |
| cggggagttt | tcgagctctc | agacgagaag | gcaacgaacc | cgatcgtgcc | ctcttttgac |
| atgagtaatg | aaggatctta | tttcttcgga | gacaatgcag | aagagtacga | caattaa |

(SEQ ID NO: 1, from GenBank Accession No. AF483604).

| | |
|---|---|
| MASQGTKRSYEQMETDGERQNATEIRASVGKMIGGIGRFYIQMCTELKLSDYEGRLIQNSLTIERMVLSA | (SEQ ID NO: 2) |
| FDERRNKYLEEHPSAGKDPKKTGGPIYRRVNGKWMRELILYDKEEIRRIWRQANNGDDATAGLTHMMIWH | |
| SNLNDATYQRTRALVRTGMDPRMCSLMQGSTLPRRSGAAGAAVKGVGTMVMELVRMIKRGINDRNFWRGE | |
| NGRKTRIAYERMCNILKGKFQTAAQKAMMDQVRESRNPGNAEFEDLTRLARSALILRGSVAHKSCLPACV | |
| YGPAVASGYDFEREGYSLVGIDPFRLLQNSQVYSLIRPNENPAHKSQLVWMACHSAAFEDLRVLSFIKGT | |
| KVLPRGKLSTRGVQIASNENMETMESSTLELRSRYWAIRTRSGGNTNQQRASAGQISIQPTFSVQRNLPF | |
| DRTTIMAAFNGNTEGRTSDMRTEIIRMMESARPEDVSFQGRGVFELSDEKAASPIVPSFDMSNEGSYFFG | |
| DNAEEYDN | |
| MASQGTKRSYEQMETDGERQNATEIRASVGKMIGGIGRFYIQMCTELKLSDYEGRLIQNSLTIERMVLSA | (SEQ ID NO: 3) |
| FDERRNKYLEEHPSAGKDPKKTGGPIYRRVNGKWMRELILYDKEEIRRIWRQANNGDDATAGLTHMDDMIWH | |
| SNLNDATYQRTRALVRTGMDPRRMCSLMQGSTLPRRRSGAAGAAVKGVGTMVMELVRMIKRGINDRNFWRGE | |
| NGRKTRIAYERMCNILKGKFQTAAQKAMMDQVRESRNPGNAEFEDLTFDDLARSALILRGSVAHKSCLPACV | |
| YGPAVASGYDFEREGYSLVGIDPFRLLQNSQVYSLIRPNENPAHKSQLVWMACHSAAFEDLRVLSFIKGT | |
| KVLPRGKLSTRGVQIASNENMETMESSTLELRSRYWAIRTRSGGNTNQQRASAGQISIQPTFSVQRNLPF | |
| DRTTIMAAFNGNTEGRTSDMRTEIIRMMESARPEDVSFQGRGVFELSDEKAASPIVPSFDMSNEGSYFFG | |
| DNAEEYDN | |
| MASQGTKRSYEQMETDGERQNATEIRASVGKMIGGIGRFYIQMCTELKLSDYEGRLIQNSLTIERMVLSA | (SEQ ID NO: 4) |
| FDERRNKYLEEHPSAGKDPKKTGGPIYRRVNGKWMRELILYDKEEIRRIWROANNGDDATAGLTHMMIWH | |
| SNLNDATYQRTRALVRTGMDPRMCSLMQGSTLPRRSGAAGAAVKGVGTMMEDDLVRMIKRGINDRNFWRGE | |
| NGRKTRIAYERMCNILKGKFQTAAQKAMMDQVRESRNPGNAEFEDLTFLARSALILRGSVAHKSCLPACV | |
| YGPAVASGYDFEREGYSLVGIDPFLLQNSQVYSLIRPNENPAHKSQLVDDWMACHSAAFEDLRVLSFIKGT | |
| KVLPRGKLSTRGVQIASNENMETMESSTLELRSRYWAIRTRSGGNTNQQRASAGQISIQPTFSVQRNLPF | |
| DRTTIMAAFNGNTEGRTSDMRTEIIRMMESARPEDVSFQGRGVFELSDEKAASPIVPSFDMSNEGSYFFG | |
| DNAEEYDN | |

Alternatively, the polypeptide is a tumor-specific antigen or tumor-associated antigen peptide, such as the MAGE family (e.g., MAGE-1, MAGE-3), MART-1, gp100, tyrosinase, tyrosinase-related protein-1, BAGE, GAGE-1, GAGE-3, gp75, oncofetal antigen, mutant p53, mutant ras or telomerase. TSA nucleic acids and polypeptides include, e.g., GenBank Accession Numbers NM_{—}004988 (human MAGE-1); NM_{—}005367 (human MAGE-12); and HSU10340 (human MAGE-2). TSA nucleic acid and polypeptide sequences are available online from the National Center for Biotechnology Information, National Library of Medicine, National Institutes of Health.

The MAGE family of genes encodes human tumor specific antigens, and various genes of this family are expressed by tumors of different histologies (melanoma, lung, colon, breast, laryngeal cancer, sarcomas, certain leukemias) and not by normal cells (generally, except testis and placenta). Wild-type MAGE-1 nucleic acid and polypeptide sequences, and modified MAGE-1 polypeptide sequences, are shown in Table 4. Hydrophobicity analysis (Kyte-Doolittle) indicates that amino acids 90-116 and 191 to 207 of SEQ ID NO: 6 contain hydrophobic domains. Disruptive elements (DD dipeptides, shown in bold) are introduced into the MAGE-1 polypeptide sequence to generated modified MAGE-1 polypeptides, as provided by SEQ ID NO: 7-8, shown in Table 4.

TABLE 4
MAGE1 nucleic acid and polypeptide sequences

Wild-type MAGE-1 nucleic acid

| | | | | | |
|---|---|---|---|---|---|
| ggatccaggc | cctgccagga | aaaatataag | ggccctgcgt | gagaacagag | ggggtcatcc |
| actgcatgag | agtggggatg | tcacagagtc | cagcccaccc | tcctggtagc | actgagaagc |
| cagggctgtg | cttgcggtct | gcaccctgag | ggcccgtgga | ttcctcttcc | tggagctcca |
| ggaaccaggc | agtgaggcct | tggtctgaga | cagtatcctc | aggtcacaga | gcagaggatg |
| cacagggtgt | gccagcagtg | aatgtttgcc | ctgaatgcac | accaagggcc | ccacctgcca |
| caggacacat | aggactccac | agagtctggc | ctcacctccc | tactgtcagt | cctgtagaat |
| cgacctctgc | tggccggctg | taccctgagt | accctctcac | ttcctccttc | aggttttcag |
| gggacaggcc | aacccagagg | acaggattcc | ctggaggcca | cagaggagca | ccaaggagaa |
| gatctgtaag | taggcctttg | ttagagtctc | caaggttcag | ttctcagctg | aggcctctca |
| cacactccct | ctctccccag | gcctgtgggt | cttcattgcc | cagctcctgc | ccacactcct |
| gcctgctgcc | ctgacgagag | tcatcatgtc | tcttgagcag | aggagtctgc | actgcaagcc |
| tgaggaagcc | cttgaggccc | aacaagaggc | cctgggcctg | gtgtgtgtgc | aggctgccac |
| ctcctcctcc | tctcctctgg | tcctgggcac | cctggaggag | gtgcccactg | ctgggtcaac |
| agatcctccc | cagagtcctc | agggagcctc | cgcctttccc | actaccatca | acttcactcg |
| acagaggcaa | cccagtgagg | gttccagcag | ccgtgaagag | gaggggccaa | gcacctcttg |
| tatcctggag | tccttgttcc | gagcagtaat | cactaagaag | gtggctgatt | tggttggttt |
| tctgctcctc | aaatatcgag | ccagggagcc | agtcacaaag | gcagaaatgc | tggagagtgt |
| catcaaaaat | tacaagcact | gttttcctga | gatcttcggc | aaagcctctg | agtccttgca |
| gctggtcttt | ggcattgacg | tgaaggaagc | agaccccacc | ggccactcct | atgtccttgt |
| cacctgccta | ggtctctcct | atgatggcct | gctgggtgat | aatcagatca | tgcccaagac |
| aggcttcctg | ataattgtcc | tggtcatgat | tgcaatggag | ggcggccatg | ctcctgagga |
| ggaaatctgg | gaggagctga | gtgtgatgga | ggtgtatgat | gggagggagc | acagtgccta |
| tggggagccc | aggaagctgc | tcacccaaga | tttggtgcag | gaaaagtacc | tggagtaccg |
| gcaggtgccg | gacagtgatc | ccgcacgcta | tgagttcctg | tggggtccaa | gggccctcgc |
| tgaaaccagc | tatgtgaaag | tccttgagta | tgtgatcaag | gtcagtgcaa | gagttcgctt |
| tttcttccca | tccctgcgtg | aagcagcttt | gagagaggag | gaagagggag | tctgagcatg |
| agttgcagcc | aaggccagtg | ggagggggac | tgggccagtg | caccttccag | ggccgcgtcc |
| agcagcttcc | cctgcctcgt | gtgacatgag | gcccattctt | cactctgaag | agagcggtca |
| gtgttctcag | tagtaggttt | ctgttctatt | gggtgacttg | gagatttatc | tttgttctct |
| tttggaattg | ttcaaatgtt | tttttttaag | ggatggttga | atgaacttca | gcatccaagt |
| ttatgaatga | cagcagtcac | acagttctgt | gtatatagtt | taagggtaag | agtcttgtgt |
| tttattcaga | ttgggaaatc | cattctattt | tgtgaattgg | gataataaca | gcagtggaat |
| aagtacttag | aaatgtgaaa | aatgagcagt | aaaatagatg | agataaagaa | ctaaagaaat |
| taagagatag | tcaattcttg | ccttatacct | cagtctattc | tgtaaaattt | ttaaagatat |
| atgcatacct | ggatttcctt | ggcttctttg | agaatgtaag | agaaattaaa | tctgaataaa |
| gaattcttcc | tgttcactgg | ctcttttctt | ctccatgcac | tgagcatctg | ctttttggaa |
| ggccctgggt | tagtagtgga | gatgctaagg | taagccagac | tcatacccac | ccatagggtc |
| gtagagtcta | ggagctgcag | tcacgtaatc | gaggtggcaa | gatgtcctct | aaagatgtag |
| ggaaaagtga | gagaggggtg | agggtgtggg | gctccgggtg | agagtggtgg | agtgtcaatg |
| ccctgagctg | gggcattttg | ggctttggga | aactgcagtt | ccttctgggg | gagctgattg |

| | | |
|---|---|---|
| taatgatctt | gggtggatcc | (SEQ ID NO: 5, from GenBank M77481) |

| | |
|---|---|
| MSLEQRSLHCKPEEALEAQQEALGLVCVQAATSSSSPLVLGTLEEVPTAGSTDPPQSPQGASAFP | (SEQ ID NO: 6) |
| TTINFTRQRQPSEGSSSREEEGPSTSCILESLFRAVITKKVADLVGFLLLKYRAREPVTKAEMLE | |
| SVIKNYKHCFPEIFGKASESLQLVFGIDVKEADPTGHSYVLVTCLGLSYDGLLGNQIMPKTGFL | |
| IIVLVMIAMEGGHAPEEEIWEELSVMEVYDGREHSAYGEPRKLLTQDLVQEKYLEYRQVPDSDPA | |
| RYEFLWGPRALAETSYVKVLEYVIKVSARVRFFFPSLREAALREEEGV | |
| MSLEQRSLHCKPEEALEAQQEALGLVCVQAATSSSSPLVLGTLEEVPTAGSTDPPQSPQGASAFP | (SEQ ID NO: 7) |
| TTINFTRQRQPSEGSSSREEEGPSTSCILESLDDFRAVITKKVADLVGFLLLKYRAREPVTKAEMLE | |
| SVIKNYKHCFPEIFGKASESLQLVFGIDVKEADPTGHSYVLVTCLGLSYDGLLGNQIMPKTGFL | |
| IIVLVMIAMEGGHAPEEEIWEELSVMEVYDGREHSAYGEPRKLLTQDLVQEKYLEYRQVPDSDPA | |
| RYEFLWGPRALAETSYVKVLEYVIKVSARVRFFFPSLREAALREEEGV | |
| MSLEQRSLHCKPEEALEAQQEALGLVCVQAATSSSSPLVLGTLEEVPTAGSTDPPQSPQGASAFP | (SEQ ID NO: 8) |
| TTINFTRQRQPSEGSSSREEEGPSTSCILESLFRAVITKKVADLVGFLLLKYRAREPVTKAEMLE | |
| SVIKNYKHCFPEIFGKASESLQLVFGIDVKEADPTGHSYVLVTCLGLSYDGLLGNQIMPKTGFLDD | |
| IIVLVMIAMEGGHAPEEEIWEELSVMEVYDGREHSAYGEPRKLLTQDLVQEKYLEYRQVPDSDPA | |
| RYEFLWGPRALAETSYVKVLEYVIKVSARVRFFFPSLREAALREEEGV | |

The modified polypeptide can be expressed from nucleic acid sequences where such sequences is DNA, RNA or any variant thereof which is capable of directing protein synthesis.

Expression Vectors Encoding Modified Polypeptides

The nucleic acid encoding the modified polypeptide is in a suitable expression vector. By suitable expression vector is meant a vector that is capable of carrying and expressing a complete nucleic acid sequence coding for the modified polypeptide. Such vectors include any vectors into which a nucleic acid sequence as described above can be inserted, along with any preferred or required operational elements, and which vector can then be subsequently introduced or transferred into a host organism and replicated in such organism. The vector can be introduced by way of transfection or infection. Preferred vectors are those whose restriction sites have been well documented and which contain the operational elements preferred or required for transcription of the nucleic acid sequence. The vectors include retroviral vectors, adenoviral vectors, lentiviral vectors, plasmid vectors, cosmid vectors, bacterial artificial chromosome (BAC) vectors, and yeast artificial chromosome (YAC) vectors.

To construct the vector of the present invention, it should additionally be noted that multiple copies of the nucleic acid sequence encoding modified polypeptide and its attendant operational elements may be inserted into each vector. In such an embodiment, the host organism would produce greater amounts per vector of the desired modified polypeptide. In a similar fashion, multiple different modified polypeptides may be expressed from a single vector by inserting into the vector a copy (or copies) of nucleic acid sequence encoding each modified polypeptide and its attendant operational elements.

Preferred vectors are those that function in a eukaryotic cell. Examples of such vectors include, but are not limited to, vaccinia virus, adenovirus or DNA constructs practiced in the art. Preferred vectors include vaccinia viruses.

Confirmation of the modification of three-dimensional structure of the polypeptide is determined by methods known in the art. For example, computer aided molecular modeling (e.g., spherical harmonics), or crystallographic analysis may be used. Alternatively, NMR or mass spectral analyses of modified polypeptides or peptide fragments thereof are performed. Further, the modified polypeptide is contacted with one or more proteolytic enzymes (e.g., proteasomal) that have differential activity (i.e., the proteolytic enzymes have a greater or reduced proteolytic activity) on the modified polypeptide in relation to the unmodified polypeptide.

The present invention provides a method of immunization comprising administering an amount of the modified polypeptide or a nucleic acid encoding the modified polypeptide (i.e., vaccine) effective to elicit a T cell response. Such T cell response can be measured by a variety of assays including ⁵¹Cr release assays (Restifo, N. P. J of Exp. Med., 177: 265-272 (1993)). The T cells capable of producing such a cytotoxic response may be CD⁸⁺ T cells, CD4+ T cells, or a population containing CD⁸⁺ T cells and CD⁴⁺ T cells.

Direct Insertion of a Disruptive Element Into the Amino Acid Sequence.

The present invention provides modified amino acids generated by insertion of a disruptive element into the primary amino acid sequence of the polypeptide. The insertion is accomplished by methods known to those skilled in the art. For example, one or more amino acids can be inserted, deleted or substituted for one or more different amino acids in a chemically synthesized polypeptide.

Administration of Nucleic Acids Encoding Modified Polypeptides

The vaccine may be administered in combination with other therapeutic ingredients including, e.g., γ-interferon, cytokines, chemotherapeutic agents, or anti-inflammatory agents.

The vaccine can be administered in a pure or substantially pure form, but it is preferable to present it as a pharmaceutical composition, formulation or preparation. Such formulation comprises a modified polypeptide or a nucleic acid encoding the modified polypeptides together with one or more pharmaceutically acceptable carriers and optionally other therapeutic ingredients. Other therapeutic ingredients include compounds that enhance antigen presentation, e.g., gamma interferon, cytokines, chemotherapeutic agents, or anti-inflammatory agents. The formulations may conveniently be presented in unit dosage form and may be prepared by methods well known in the pharmaceutical art.

Formulations suitable for intravenous, intramuscular, subcutaneous, or intraperitoneal administration conveniently comprise sterile aqueous solutions of the active ingredient with solutions which are preferably isotonic with the blood of the recipient. Such formulations may be conveniently prepared by dissolving solid active ingredient in water containing physiologically compatible substances such as sodium chloride (e.g., 0.1-2.0M), glycine, and the like, and having a buffered pH compatible with physiological conditions to produce an aqueous solution, and rendering said solution sterile. These may be present in unit or multi-dose containers, for example, sealed ampoules or vials.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057).

The formulations of the present invention may incorporate a stabilizer. Illustrative stabilizers are polyethylene glycol, proteins, saccharide, amino acids, inorganic acids, and organic acids which may be used either on their own or as admixtures. Two or more stabilizers may be used in aqueous solutions at the appropriate concentration and/or pH. The specific osmotic pressure in such aqueous solution is generally in the range of 0.1-3.0 osmoses, preferably in the range of 0.80-1.2. The pH of the aqueous solution is adjusted to be within the range of 5.0-9.0, preferably within the range of 6-8.

When oral preparations are desired, the compositions may be combined with typical carriers, such as lactose, sucrose, starch, talc magnesium stearate, crystalline cellulose, methyl cellulose, carboxymethyl cellulose, glycerin, sodium alginate or gum arabic among others.

The method of immunization may comprise administering a nucleic acid sequence capable of directing host organism production of the modified polypeptide in an amount effective to elicit a T cell response. Such nucleic acid sequence may be inserted into a suitable expression vector by methods known to those skilled in the art. Expression vectors suitable for producing high efficiency gene transfer in vivo include retroviral, adenoviral and vaccinia viral vectors. Operational elements of such expression vectors are known to one skilled in the art. A preferred vector is vaccinia virus.

Expression vectors containing a nucleic acid sequence encoding modified polypeptide can be administered intravenously, intramuscularly, subcutaneously, intraperitoneally or orally. A preferred route of administration is intravenous.

The modified polypeptides and expression vectors containing nucleic acid sequence capable of directing host organism synthesis of modified polypeptides may be supplied in the form of a kit, alone, or in the form of a pharmaceutical composition.

Expression vectors include one or more regulatory sequences, including promoters, enhancers and other expression control elements (e.g., polyadenylation) signals. Such regulatory sequences are described, for example, in Goeddel, GENE EXPRESSION TECHNOLOGY: METHODS IN ENZYMOLOGY 185, Academic Press, San Diego, Calif. (1990).

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

The invention also provides a vaccine for immunizing a mammal against cancer, viral infection, bacterial infection, parasitic infection, or autoimmune disease, comprising a modified polypeptide or an expression vector containing nucleic acid sequence capable of directing host organism synthesis of modified polypeptide in a pharmaceutically acceptable carrier. In an alternative embodiment, multiple expression vectors, each containing nucleic acid sequence capable of directing host organism synthesis of different modified polypeptides, may be administered as a polyvalent vaccine.

Vaccination can be conducted by conventional methods. For example, a modified polypeptide can be used in a suitable diluent such as saline or water, or complete or incomplete adjuvants. The vaccine can be administered by any route appropriate for eliciting T cell response, such as intravenous, intraperitoneal, intramuscular, and subcutaneous. The vaccine may be administered once or at periodic intervals until a T cell response is elicited. T cell response may be detected by a variety of methods known to those skilled in the art, including but not limited to, cytotoxicity assay, proliferation assay and cytokine release assays.

The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient.

The present invention also includes a method for treating cancer, viral infection, bacterial infection, parasitic infection, disorders associated with altered gene expression such as cell proliferative disorders or autoimmune disease, by administering pharmaceutical compositions comprising a modified polypeptide or an expression vector containing nucleic acid sequence capable of directing host organism synthesis of a modified polypeptide in a therapeutically effective amount. Again as with vaccines, multiple expression vectors may also be administered simultaneously. When provided therapeutically, the modified polypeptide or modified polypeptide-encoding expression vector is provided at (or after) the onset of the infection or at the onset of any symptom of infection or disease caused by cancer, a virus, a bacteria, a parasite, a prion, or autoimmune disease. The therapeutic administration of the modified polypeptide or modified polypeptide-encoding expression vector serves to attenuate the infection or disease.

A preferred embodiment is a method of treatment comprising administering a vaccinia virus containing nucleic acid sequence encoding modified polypeptide to a mammal in therapeutically effective amount.

### EXAMPLES

### Example 1

### Construction of Plasmids and Vaccinia Virus Recombinants

The plasmids were constructed containing NP-genes as indicated in Table 1. These plasmids were utilized to construct recombinants of vaccinia virus (VVR) expressing “stable” and “destabilized” NP-antigens for DNA vaccination. (Table 2) The protein was destabilized using the C-end motif of ornithyn-decarboxylase (Clontech).

TABLE 1
Plasmids Constructed

| Final | | | | |
|---|---|---|---|---|
| plasmid | Basic plasmid | Gene inserted | Promoter | Use |
| pNP (5.5 kb) | pd1EGFP-N1 | IVA NP gene | CMV | DNA |
| pdNP | pd1EGFP-N1 | (pCMV- | CMV | vaccination |
| (5.7 kb) | | PR8NPORF) | | Insertion |
| pNP65 | pSC65 | | VV-P65 | into vaccinia |
| (8.8 kb) | | | | viral vectors |
| pdNP65 | pSC65 | | VV-P65 | |
| (9.0 kb) | | | | |

TABLE 2
List of VVR constructed

| | Gene inserted | | |
|---|---|---|---|
| | into tk-gene of VV | | |
| Recombinants | (WR strain) | Expression | Destabilization |
| W-NP | NP | + | |
| W-dNP | DNP | + | − |

### Example 2

### Expression and Proteolytic Stability of NP-Protein Cloned in Vaccinia Virus Recombinants

CV1 cells were inoculated with W—NP or W-dNP recombinants (I bfu/cell). 40 hours later, the cells were treated with 40 μg/ml of cycloheximide and incubated for 8 more hours. The cells were collected and homogenized, and protein content was tested by Western Blot on the level of NP-protein. The Western Blot results indicate that both recombinants were actively expressing NP-protein in its native sequence, and containing C-end motif (dNP). Fusion with C-end motif did not lead to any significant increase in proteolytic processing of dNP. Both NP and dNP were readily ubiquitinated possessing triple bands on the Western Blot, the tight globular 3-D conformation prevented the protein from proteasome processing.

### Example 3

### Protective Immune Response of W-NP and W-dNP Recombinants

To test the protective immune response, Balb/c mice were immunized twice with corresponding VVR strains and infected with influenza A virus (IVA). Balb/c mice were infected with influenza A virus A/Aichi 2/68 (N3H2). The results depicted in Table 3 indicate that NP-protein delivered via VVR vector is an effective protector against influenza virus A infection. Importantly, the strain used for infection was a remote viral strain to the one NP-protein was cloned from. It indicates that T-antigenic vaccination by NP-protein protects against wide-range of influenza A strains.

TABLE 3
Immunogenicity of VVR W-NP and W-dNP against
influenza virus (A/Aichi2/68) infection in mice

Dilution of infecting
IVA (A/Aichi2/68 strain)

| Immunizing virus | 10⁰ | 10⁻¹ | 10⁻² | 10⁻³ | IgLD50 |
|---|---|---|---|---|---|
| W-NP | | 13/18 | 1/19 | 0/17 | 1.3 |
| W-dNP | | 10/17 | 0/17 | 0/16 | 1.1 |
| WR | | 8/11 | 4/6 | 1/6 | 2.0 |
| None | 10/11 | 9/11 | 4/11 | 0/12 | 1.7 |

### Example 4

### In Silico Generation of Influenza Vaccines

Improved influenza vaccines may be generated as follows. The three-dimensional structure of an influenza polypeptide (e.g., NP or hemagglutinin (HA)) or a portion thereof is determined by molecular modeling, crystallography, or other means known to one of ordinary skill in the art. One or more disruptive elements are introduced into the primary amino acid sequence of the protein (see, e.g., the modified NP peptides disclosed in Table 1), and the effect(s) of these elements on the three-dimensional structure are determined as above. In embodiments of the invention, a disruptive element is placed within an alpha helical region of the polypeptide, such that said alpha helical region is disrupted. Alternatively, a disruptive element may be introduced such that the modified polypeptide becomes a substrate for a protease that does not act upon the unmodified protein. The modified and unmodified polypeptides are expressed in cultured cells and their stability is quantified by standard assays.

### Example 5

### Use of Modified Influenza NP Polypeptides to Increase Antigen Presentation

The influenza NP polypeptide sequence has a primarily α-helical structure with just a few β-strands. Secondary structure analyses indicate that the NP polypeptide is approximately 39% α-helical, 16% α-strands, and 45% loops and turns. Moreover, the NP polypeptide is a globular protein (216 out of 498 amino acids are predicted to be exposed.) One helical region of the NP polypeptide is from amino acids 256 to 261 of SEQ ID NO:2, with only amino acid residue 261 predicted to be exposed on the protein surface. Thus, amino acids 256 and 257 (LT) are targets for replacement by two aspartate residues (DD). This targeted mutation is performed using PCR-based mutagenesis on the NP nucleic acid. The resulting modified NP nucleic acid is cloned into an expression vector, which is introduced into host cells. The expressed modified NP is expressed, and the proteolytic degradation of the modified polypeptide is compared with the expressed wild type NP polypeptide. The expressed modified NP polypeptide is contacted with antigen presenting cells (APCs) such as B cells, macrophages or dendritic cells, and the increased presentation of fragments of modified NP polypeptide is determined in reference to wild type NP polypeptide contacted with APCs.

Previous studies conducted by others have shown a degree of enhancement of NP protein degradation in cells by including a sequence in external portions of NP protein that enhances ubiquitination. (See, e.g., Gschoesser et al., 2002 Vaccine 20: 3731-38; Anton et al., 1999 J. Cell Biol. 146:113-124; Anton et al., 1998 J Immunol. 160(10):4859-68; and Cerundolo et al., 1997 Eur. J. Immunol. 27:33641). This degree of degradation resulted in a nominal degree of better antigenic presentation for development of an immune response.

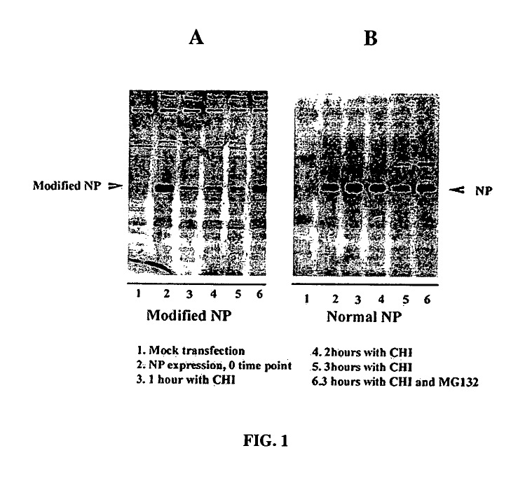

A modified NP polypeptide was created from a modified NP nucleic acid by inserting a nucleic acid sequence encoding the dipeptide sequence DD such that these two amino acids were inserted between M136 and M137 of SEQ ID NO: 2. The modified NP nucleic acid sequence was inserted into a vector containing a FLAG-tag under the regulation of a CMV promoter. HeLa cells were transiently transfected with either the modified NP vector, or a vector encoding the unmodified NP polypeptide, or mock-transfected. After 48 hours, the transfected cells were treated with an inhibitor of protein synthesis, cycloheximide (CHI) or a combination of CHI and an inhibitor of proteasome MG132. Untreated cells served as a control. Cells were lysed after 1, 2, or 3 hours, and the cell lysates were subjected to polyacrylamide gel electrophoresis followed by immunoblotting with an anti-FLAG antibody. As shown in FIG. 1a, cells expressing a modified NP polypeptide in the presence of CHI have substantially less full-length NP polypeptide (indicated by arrowhead) than either modified NP-expressing cells not exposed to CHI or cells expressing non-modified (“normal NP”) NP polypeptide, in the presence or absence of CHI. Notably, incubation of modified NP polypeptide for 3 hours in the presence of CHI and the protease inhibitor MG132, blocks proteolysis of the modified NP polypeptide.

### Example 6

### Generation, Expression and Proteolytic Stability of Modified NP Proteins Cloned Into Vaccinia Viral Vectors

The Influenza A nucleoprotein gene (e.g., SEQ ID NO: 1, which corresponds to the Influenza A virus strain A/Paris/908/97(H3N2)) is subjected to directed mutagenesis to insert a disruptive element, such as PCR-based mutagenesis, such that a modified nucleic acid is generated. The modified nucleic acid encodes for a modified NP polypeptides (e.g., SEQ ID Nos 3-4). The modified nucleic acid is cloned into a vector, such as a vaccinia viral vector (e.g., modified vaccinia virus Ankara vectors), or a plasmid expression vector (e.g., pcDNA3 (Invitrogen)) used to generate vaccinia virus recombinants, capable of expressing modified NP polypeptides (mNP) or wild-type (unmodified) NP polypeptides (Wt-NP), or recombinant DNA for DNA vaccination. In certain embodiments, the modified nucleic acid is cloned into an epitope tagging vector such that the NP polypeptide is expressed as a fusion protein containing an immunogenic epitope such as FLAG, c-myc, or poly-His (6×-His).

Epithelial cells (e.g., the CV1 cell line) are inoculated with mNP or WtNP recombinants (1 burst-forming unit (bfu) per cell). After 40 hours, the cells are treated with 40 μg/ml of cycloheximide and incubated for 8 more hours. The cells are collected and homogenized, and expressed protein content is determined by Western blotting on the level of mNP and WtNP polypeptides. The Western Blot results indicate that introduction of a disruptive element (e.g., DD) into NP leads to a significant increase in proteolytic processing of the NP polypeptide.

To measure the protective immune response, Balb/c mice are immunized twice with the nucleic acid recombinants or vaccinia virus recombinants encoding either modified NP or WtNP. Mice immunized twice with nucleic acid vectors or recombinant vaccinia virus vectors containing wild-type NP nucleic acids virus are used as control. After six weeks, Balb/c mice are infected with influenza A virus A/Aichi 2/68 (N3H2), although other strains such as strain A/Paris/908/97(H3N2) are contemplated. The modified NP-protein delivered via a VVR vector is a more effective protector against influenza virus A infection, as compared to the wild-type NP protein. The increased survival of mice immunized with mNP, as compared to mice immunized with WtNP, indicates that the mNP is protective agains influenza virus. Notably, the A/Aichi 2/68 (N3H2) strain used for infection is distinct from the strain from which the NP-protein was cloned. Therefore, the vaccination by modified NP protein protects against a wide range of influenza A strains.

### Example 7

### Generation and Use of Modified MAGE-1 Polypeptides to Increase Antigen Presentation

Expression of the MAGE-1 polypeptide has been associated with cancer, including melanoma. The MAGE polypeptide sequence has numerous hydrophobic domains. A wild-type MAGE-1 polypeptide is provided in SEQ ID NO: 6. Based on the polypeptide structure, the region including amino acids 191-207 is a target for insertion of two aspartate residues (DD), or the replacement of two or more amino acids with aspartate residues. This targeted mutation is performed using PCR-based mutagenesis on the MAGE-1 nucleic acid (e.g., the nucleic acid sequence provided as SEQ ID NO: 5). The resulting modified MAGE-1 nucleic acid is cloned into an expression vector, which is introduced into host cells. In embodiments, the modified MAGE-1 nucleic acid sequence is inserted into a vector containing an epitope tag (e.g., a FLAG-tag) under the regulation of a promoter. The promoter may be a constitutive promoter or an inducible promoter, as known by one skilled in the art. The inducible promoter allows expression of the modified MAGE-1 nucleic acid to be turned on and off as required. The expressed modified MAGE-1 is expressed, and the proteolytic degradation of the modified polypeptide is compared with the expressed wild type MAGE-1 polypeptide. The expressed modified MAGE-1 polypeptide is contacted with antigen presenting cells (APCs) such as macrophages or dendritic cells, and the increased presentation of fragments of modified MAGE-1 polypeptide is determined in reference to wild type MAGE-1 polypeptide contacted with APCs.

A mammalian subject (e.g., a human patient) is identified as having cancer or having an increased suceptibility to cancer (such as melanoma), as determined by genetic and/or other diagnostic tests known to one skilled in the art. A modified MAGE-1 nucleic acid in a vector suitable for administration to a mammal is provided to the subject, such that proteolytic degradation of the modified MAGE-1 polypeptide encoded by the modified MAGE-1 nucleic acid is increased, relative to the wild-type (unmodified) MAGE-1 polypeptide. This increase in proteolysis results in increased antigen presentation, and increased clearance (e.g., destruction) of cells expressing the MAGE-1 polypeptide (either the wild-type MAGE-1 polypeptide or a mutant thereof). Thus, the present invention provides a method for treating a subject having cancer or having an increased suceptibility to cancer, using modified TSA or TAA nucleic acids and polypeptides, as described above.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of inducing an immune response in a subject against a protein, comprising introducing a modified protein into said subject, wherein said modified protein includes a disruptive element, wherein said disruptive element is located in an internal region of said modified protein, such that the immune response is induced.

2. The method of claim 1, wherein said modified polypeptide has altered susceptibility to proteolysis as compared to an unmodified protein.

3. The method of claim 1, wherein said disruptive element is located in a domain structure of said protein.

4. The method of claim 1, wherein said internal region of said amino acid sequence is hydrophobic.

5. The method of claim 1, wherein said disruptive element is located in an amphiphilic α-helical region.

6. The method of claim 1, wherein said disruptive element comprises one or more hydrophilic amino acids substituted for one or more hydrophobic amino acids.

7. The method of claim 6, wherein said hydrophobic amino acids are selected from the group consisting of phenylalanine, cysteine, isoleucine, leucine, valine and tryptophan.

8. The method of claim 6, wherein said hydrophilic amino acids are selected from the group consisting of aspartate, asparagine, glutamate, glutamine, lysine, or arginine.

9. The method of claim 6, wherein said disruptive element comprises one to ten hydrophilic amino acids.

10. The method of claim 6, wherein said one or more hydrophilic amino acids are contiguous.

11. The method of claim 1, wherein said protein is selected from the group consisting of a viral protein, a tumor-associated polypeptide, a cell proliferative disorder-associated polypeptide, and a disease-associated polypeptide.

12. The method of claim 1, wherein said polypeptide is a viral core protein.

13. The method of claim 12, wherein said viral core protein is a NP protein.

14. The method of claim 1, wherein said modified protein, when undergoing proteolytic processing, is degraded to a peptide less than about 50 amino acids in length compared to an unmodified viral protein.

15. The method of claim 1, wherein said modified viral protein, when undergoing proteolytic processing, is degraded to a peptide less than about 25 amino acids in length compared to an unmodified viral protein.

16. The method of claim 1, wherein said modified viral protein, when undergoing proteolytic processing, is degraded to a peptide between 5-10 amino acids in length compared to an unmodified viral protein.

17. The method of claim 1, wherein said disruptive element is a dipeptide.

18. The method of claim 17, wherein said dipeptide is aspartate-aspartate

19. The method of claim 1, wherein said disruptive element alters the tertiary structure of said modified viral protein as compared to wild-type or unmodified viral protein.

20. The method of claim 1, wherein said response is a T cell response.

21. A vaccine comprising, in an amount effective to elicit an immune response, a nucleic acid molecule encoding a modified viral protein, wherein said modified protein includes a disruptive element, wherein said disruptive element is located in an internal region of said modified viral protein, wherein said nucleic acid molecule is capable of being expressed.

22. The vaccine of claim 21, wherein said modified viral protein has altered susceptibility to proteolysis as compared to an unmodified viral protein.

23. The vaccine of claim 21, wherein said nucleic acid molecule is operably linked to a promoter.

24. The vaccine of claim 21, wherein said nucleic acid molecule is in a vector.

25. The vaccine of claim 24, wherein said vector is a vaccinia virus vector.

26. The vaccine of claim 24, wherein said modified polypeptide has altered susceptibility to proteolysis as compared to an unmodified viral protein.

27. The vaccine of claim 26, wherein said disruptive element is located in a domain structure of said modified viral protein.

28. The vaccine of claim 21, wherein said internal region of said amino acid sequence comprises one or more hydrophobic amino acids.

29. The vaccine of claim 21, wherein said disruptive element is located in an amphiphilic α-helical region.

30. The vaccine of claim 21, wherein said disruptive element comprises one or more hydrophilic amino acids substituted for one or more hydrophobic amino acids.

31. The vaccine of claim 30, wherein said hydrophobic amino acids are selected from the group consisting of phenylalanine, cysteine, isoleucine, leucine, valine and tryptophan.

32. The vaccine of claim 30, wherein said hydrophilic amino acids are selected from the group consisting of aspartate, asparagine, glutamate, glutamine, histidine, lysine, or arginine.

33. The vaccine of claim 30, wherein said disruptive element comprises between one and about ten hydrophilic amino acids.

34. The vaccine of claim 33, wherein said one or more hydrophilic amino acids are contiguous.

35. The vaccine of claim 21, wherein said protein is an influenza viral protein.

36. The vaccine of claim 21, wherein said polypeptide is a viral core protein.

37. The vaccine of claim 21, wherein said viral core protein is a NP protein.

38. The vaccine of claim 21, wherein said modified viral protein, when undergoing proteosomal processing, is degraded to a peptide less than 50 amino acids in length compared to said corresponding unmodified polypeptide.

39. The vaccine of claim 21, wherein said modified viral protein, when undergoing proteosomal processing, is degraded to a peptide less than 25 amino acids in length compared to said corresponding unmodified polypeptide.

40. The vaccine of claim 21, wherein said modified viral protein, when undergoing proteosomal processing, is degraded to a peptide between 5-10 amino acids in length compared to said corresponding unmodified viral polypeptide.

41. The vaccine of claim 21, wherein said peptide binds an MHC class I molecule.

42. A method of inducing an immune response in a subject against a protein, comprising introducing into a subject a nucleic acid molecule encoding a modified protein, wherein said modified protein contains a disruptive element, wherein said disruptive element is located in an internal region of said modified protein, when said nucleic acid molecule is capable of being expressed in a cell, such that the immune response is induced.

43. A method of immunization, comprising administering to a subject the vaccine of claim 21.

44. The method of claim 43, where said administration is by a route selected from the group consisting of intraperitoneal, subcutaneous, nasal, intravenous, oral, topical and transdermal delivery.

45. The method of claim 43, wherein said vaccine is administered in a vector or a liposome.

46. The method of claim 45, wherein said vector is a viral vector, DNA vector, or an RNA vector.

47. The method of claim 43, wherein said subject is further administered a compound that is selected from the group consisting of a compound that increases antigen presentation, an adjuvant, and a cytokine.

48. The method of claim 47, wherein said compound is interferon-γ.

49. The method of claim 43, wherein said subject is suffering from or at risk of cancer, a viral infection or a disorder associated with improper gene expression.

50. A method of immunization, comprising:
a) providing a subject cell;
b) contacting said cell with the vaccine of claim 21; and
c) administering said cell to the subject, such that said subject is immunized thereby.

51. A vaccine comprising, in an amount effective to elicit an immune response, a vector comprising a nucleic acid molecule encoding a modified NP polypeptide, wherein said modified NP polypeptide includes a disruptive element, wherein said disruptive element is located in an internal region of said modified NP protein, wherein said nucleic acid molecule is operably linked to a promoter.

52. The vaccine of claim 51, wherein said promoter is a CMV promoter or a VV-P65 promoter.

53. The vaccine of claim 52, wherein said vector is a vaccinia virus vector.

54. A method of forming a vaccine capable of stimulating the immune mechanism of a mammal, comprising introducing a disruptive element into a nucleic acid encoding a polypeptide to form a modified polypeptide, wherein said disruptive element is located in an internal region of said modified protein, wherein said modified polypeptide has altered susceptibility to proteolysis as compared to an unmodified protein, and combining said modified polypeptide with a vaccine carrier, such that a vaccine is formed.

55. A method of forming a vaccine capable of stimulating the immune mechanism of a mammal, comprising introducing a disruptive element into a polypeptide to form a modified polypeptide, wherein said disruptive element is located in an internal region of said modified protein, wherein said modified polypeptide has altered susceptibility to proteolysis as compared to an unmodified protein, and combining said modified polypeptide with a vaccine carrier, such that a vaccine is formed.

56. A method of immunization, comprising:
a) providing a subject cell;
b) contacting said cell with the vaccine of claim 21; and
c) administering said cell to the subject, such that said subject is immunized thereby.

57. A method of generating a substantially pure population of educated, antigen-specific immune effector cells, comprising contacting immune effector cells with an antigen presenting cell, wherein said antigen presenting cell contains a nucleic acid molecule encoding a modified protein, wherein said modified protein contains a disruptive element, wherein said disruptive element is located in an internal region of said modified protein, wherein said nucleic acid molecule is capable of being expressed in said antigen presenting cell.

58. A substantially pure population of educated, antigen-specific immune effector cells produced by culturing immune effector cells with an antigen presenting cell, wherein said antigen presenting cell contains a nucleic acid molecule encoding a modified protein, wherein said modified protein contains a disruptive element, wherein said disruptive element is located in an internal region of said modified protein, wherein said nucleic acid molecule is capable of being expressed in said antigen presenting cell.

59. The population of claim 58, wherein said antigen-specific immune effector cells are T lymphocytes.

60. A method of inducing an immune response in a subject against a protein, comprising introducing a modified protein into said subject wherein said modified protein includes a disruptive element, wherein said disruptive element is located in an internal region of said modified protein, wherein said modified protein further includes a modification site, such that the immune response is induced.

61. The method of claim 60, wherein said modification site is a site for a biological process that is selected from the group consisting of phosphorylation, dephosphorylation, glycosylation, acetylation, methylation, ubiquitination, sulfation, proteolysis, prenylation, and selenium incorporation.

62. The method of claim 61, wherein said biological process causes an alteration in the tertiary structure of said protein.

63. A modified peptide comprising the amino acid sequence of SEQ ID NO: 3.

64. A nucleic acid molecule comprising a nucleic acid sequence encoding the amino acid sequence of SEQ ID NO: 4.

65. A method for presentation of antigens, comprising:
a) contacting an antigen presenting cell with a nucleic acid molecule, said nucleic acid molecule encoding a polypeptide having a disruptive element, wherein said disruptive element is located in an internal region of said protein, and
b) causing said nucleic acid molecule to be expressed in said antigen presenting cell, such that one or more polypeptides derived from said polypeptide are presented as antigens by said antigen presenting cell.

66. A method for formulation of a vaccine, comprising:
a) providing an amino acid sequence encoding a viral protein;
b) identifying one or more amino acids of said polypeptide that are capable of being disrupted by the introduction of a disruptive element, such that said disruption alters the tertiary structure of said polypeptide;
c) introducing said disruptive element into a nucleic acid sequence encoding said protein, wherein said nucleic acid is capable of being expressed,
whereby a vaccine is formulated.
